(19) United States
(12) Patent Application Publication
(10) Pub. No.: US 2017/0340295 A1
(43) Pub. Date: Nov. 30, 2017

(54) BIPLANE IMAGING SYSTEM AND METHOD OF QUARTER SCAN THREE-DIMENSIONAL IMAGING

(71) Applicant: Whale Imaging, Inc., Waltham, MA (US)

(72) Inventors: Xun Zhu, Waltham, MA (US); Xingbai He, Belmont, MA (US); Changguo Ji, Lexington, MA (US)

(21) Appl. No.: 15/596,926

(22) Filed: May 16, 2017

Related U.S. Application Data
(60) Provisional application No. 62/392,322, filed on May 27, 2016.

(51) Int. Cl. A61B 6/03; A61B 6/00

(52) U.S. Cl. CPC A61B 6/032; A61B 6/54; A61B 6/487; A61B 6/466; A61B 6/4007; A61B 6/4085; A61B 6/4078; A61B 6/4429

(57) ABSTRACT

A method and apparatus for performing three dimensional (3D) paradoxical pulse bi-planar synchronous real-time imaging. In a 3D imaging scan mode, the bi-planar imaging method and apparatus of the present invention executes a cross angle of two X-ray imaging subsystems with a sweeping angle of the two subsystems to be configured with a mechanical offset of 90 degree plus a half-fan beam angle of the X-ray beam.

### CROSS-REFERENCE TO RELATED APPLICATION(S)

This application claims priority to, and the benefit of, co-pending U.S. Provisional Application No. 62/392,322, filed May 27, 2016, for all subject matter common to both applications. The disclosure of said provisional application is hereby incorporated by reference in its entirety.

### FIELD OF THE INVENTION

The present invention relates to a system and method for implementing cone beam computed tomography suitable for capturing three dimensional imaging of a subject. In particular, the present invention relates to a cone beam computed tomography system capable of provide paradoxical pulse bi-planar synchronous real-time imaging.

### BACKGROUND

Generally, when conducting operations such as bone fixation operations, surgeons often need to observe the precise location of metallic implants or mobile surgical instruments for more precise positioning during the operations. There are different methods and systems that can be implemented to perform real-time imaging or interventional radiology during such procedures. Cone Beam Computed Tomography (or CBCT, also referred to as C-arm CT, flat panel CT in fluoroscopic imaging, etc.) is medical imaging technique that can be implemented in real-time by implementing X-ray computed tomography where the X-rays are divergent, forming a cone. CBCT has become increasingly important in diagnosis, surgical planning and validation in interventional radiology and other imaging applications. Additionally, three dimensional (3D) imaging methods using CBCT is becoming an important tool for patient positioning and verification. Typically the three dimensional imaging methods are implemented using a single plane C-arm fluoroscopy system with a combination of X-ray image intensifiers (XRII) or flat panel detectors (FPD).

Another common technique for real time imaging is Conventional Multi-Detector Row CT (MDCT). MDCT has evolved into clinical practice with a rapid increase of the number of detector slices. U.S. Patent Application Publication No. 2014/0247917 describes a modified a dual source CT (DSCT). The modified DSCT is a CT scanner with two X-ray tubes and two detectors (mounted on a CT gantry with a mechanical offset of 90°) that has the potential to overcome limitations of conventional MDCT systems, such as temporal resolution for cardiac imaging. A dual source CT scanner, such as the CT scanner disclosed in U.S. Patent Application Publication No. 2014/0247917, provides temporal resolution equivalent to a quarter of the gantry rotation time of 360 degrees. In addition to the benefits for cardiac scanning, the dual source CT scanner also enables functionality beyond conventional CT imaging by obtaining dual energy information if the two tubes are operated at different voltages.

### SUMMARY

There is a need for improved three dimensional paradoxical pulses bi-planar synchronous real-time imaging utilizing cone beam computed tomography (CBCT). The present invention is directed toward further solutions to address this need, in addition to having other desirable characteristics. Specifically, the present invention is directed to a three dimensional (3D) paradoxical pulse bi-planar synchronous real-time imaging method and system. In a 3D imaging scan mode, the bi-planar imaging device of the present invention executes a cross angle of two X-ray imaging subsystems with a sweeping angle of the two subsystems to be configured with a mechanical offset of 90 degree plus a half-fan beam angle of the X-ray beam. In particular, the present invention enables the capability of cone beam computed tomography with biplane fluoroscopic imaging by quarter scan executed by rotating two imaging assemblies on gantry by 90 degree plus half fan beam angle or 90 degree plus whole fan beam angle created by an energy emitting device. The 90 degree plus half fan beam angle is dictated by the offset angle of two imaging assemblies.

In accordance with example embodiments of the present invention, a method for conducing three-dimensional cone beam computed tomography imaging with a bi-planar imaging device is provided. The method includes initializing a bi-planar imaging device. The device includes a support gantry having a generally arc shape about an interior center focus point with a first terminal end and a second terminal end. The device also includes a first imaging assembly positioned on the support gantry and configured to rotate along the generally arc shape of the support gantry, the first imaging assembly comprising a first imaging energy emitter positioned opposite a first imaging receptor, wherein one of the first imaging energy emitter or the first imaging receptor is positioned at the first terminal end of the support gantry. The device further includes a second imaging assembly positioned on the support gantry, the second imaging assembly having a second imaging energy emitter positioned opposite a second imaging receptor, wherein one of the second imaging energy emitter or the second imaging receptor is positioned at the second terminal end of the support gantry and a control unit that directs movement and positioning of the support gantry. The method also includes positioning the first imaging assembly and the second imaging assembly at locations to create an offset angle between the first imaging receptor of the first imaging assembly and the second imaging energy emitter of the second imaging assembly with a mechanical offset of 90 degrees plus half a fan beam angle produced by energy emissions of the first imaging energy emitter and the second imaging energy emitter. The method further includes activating the bi-planar imaging device with a subject patient positioned between the first imaging assembly and the second imaging assembly, obtaining, by the first imaging receptor and the second imaging receptor, raw image data of the subject patient, and communicating the raw image data to a processing and display device. The method also includes transforming the raw image data of the subject patient, by the processing and display device, into a three-dimensional image of the subject patient and displaying the three-dimensional image on a display.

In accordance with aspects of the present invention, positioning the first imaging assembly causes one of the first imaging energy emitter or the first imaging receptor positioned at the first terminal end of the support gantry to rotate between the second imaging energy emitter and the second imaging receptor of the second imaging assembly at an offset angle between the first imaging receptor and the second imaging receptor of 0 degrees to 180 degrees.

In accordance with aspects of the present invention, the first imaging assembly is positioned and oriented to emit imaging energy in an LT plane and the second imaging assembly is positioned and oriented to emit imaging energy in an AP plane, perpendicular to the LT plane. The first imaging assembly can be positioned and oriented to emit imaging energy in an AP plane and the second imaging assembly is positioned and oriented to emit imaging energy in an LT plane, perpendicular to the AP plane.

In accordance with aspects of the present invention, the first imaging receptor and the second imaging receptor are one of an image intensifier or a flat panel detector. The first imaging energy emitter and the second imaging energy emitter can be X-ray sources configured to produce X-ray beams.

In accordance with aspects of the present invention, the bi-planar imaging device is one of a ceiling or flooring mounted dual plane fluoroscopic system.

In accordance with example embodiments of the present invention, a bi-planar imaging apparatus is provided. The apparatus includes a support gantry having a generally arc shape about an interior center focus point with a first terminal end and a second terminal end. The apparatus also includes a first imaging assembly positioned on the support gantry and configured to rotate along the generally arc shape of the support gantry, the first imaging assembly including a first imaging energy emitter positioned opposite a first imaging receptor, wherein one of the first imaging energy emitter or the first imaging receptor is positioned at the first terminal end of the support gantry. The apparatus further includes a second imaging assembly positioned on the support gantry, the second imaging assembly including a second imaging energy emitter positioned opposite a second imaging receptor, wherein one of the second imaging energy emitter or the second imaging receptor is positioned at the second terminal end of the support gantry. The apparatus also includes a control unit that directs movement and positioning of the support gantry. The rotation of the first imaging assembly causes the one of the first imaging energy emitter or the first imaging receptor positioned at the first terminal end of the support gantry to rotate between the second imaging energy emitter and the second imaging receptor of the second imaging assembly at an offset angle between the first imaging receptor and the second imaging receptor of 0° to 180°. Additionally, the apparatus performs a three-dimensional image scan by positioning of the first imaging assembly and the second imaging assembly at locations to create the offset angle between the first imaging receptor of the first imaging assembly and the second imaging energy emitter of the second imaging assembly with a mechanical offset of 90 degree plus half a fan beam angle produced by energy emissions of the first imaging energy emitter and the second imaging energy emitter.

In accordance with aspects of the present invention, the first imaging assembly is positioned and oriented to emit imaging energy in an LT plane and the second imaging assembly is positioned and oriented to emit imaging energy in an AP plane, perpendicular to the LT plane. The first imaging assembly can be positioned and oriented to emit imaging energy in an AP plane and the second imaging assembly is positioned and oriented to emit imaging energy in an LT plane, perpendicular to the AP plane.

In accordance with aspects of the present invention, the first imaging receptor and the second imaging receptor are one of an image intensifier or a flat panel detector. The first imaging energy emitter and the second imaging energy emitter can be X-ray sources configured to produce X-ray beams.

In accordance with aspects of the present invention, the bi-planar imaging apparatus is mounted on a G-arm system.

### BRIEF DESCRIPTION OF THE FIGURES

These and other characteristics of the present invention will be more fully understood by reference to the following detailed description in conjunction with the attached drawings, in which:

FIG. 1 depicts the main components of a conventional C-arm apparatus, which can be configured with a CBCT scanner;

FIGS. 2A and 2B depict example embodiments of a mobile bi-planar imaging device, in accordance with the present invention;

FIG. 3 is a flowchart depicting an example operation of the mobile bi-planar imaging device of the present invention; and

FIG. 4 is a diagrammatic illustration of a high level architecture for implementing processes in accordance with aspects of the invention.

### DETAILED DESCRIPTION

An illustrative embodiment of the present invention relates to a cone beam computed tomography system making it possible to provide paradoxical pulse bi-planar synchronous real-time imaging. Specifically, the present invention relates to a G-arm support gantry configured to perform CBCT medical imaging utilizing rotatable imaging assemblies. The imaging assemblies are rotatable around the gantry of the G-arm and are configured to traverse around the gantry between 0 and 180 degrees of rotation. The rotatable imaging assemblies enable the system of the present invention to capture real-time three-dimensional imaging data of a subject center between the imaging assemblies. In particular, the imaging assembly structures are enabled in accordance with the novel inventive configuration to rotate at a mechanical offset of 90 degrees plus a distance of one half of a fan beam angle produced by the energy emitters of the imaging assemblies, without negatively impacting other functionality or capabilities of the system. This mechanical offset enables a user of the present invention to limit rotation of the imaging assemblies to a quarter scan, thus reducing effort and time required during a procedure. Because the Federal Drug Administration regulates the maximum angular rotation speeds for such methodologies (e.g., CBCT), the quarter scan implementation enabled by the present invention can reduce scan time by half when compared with the one plane half scan configuration.

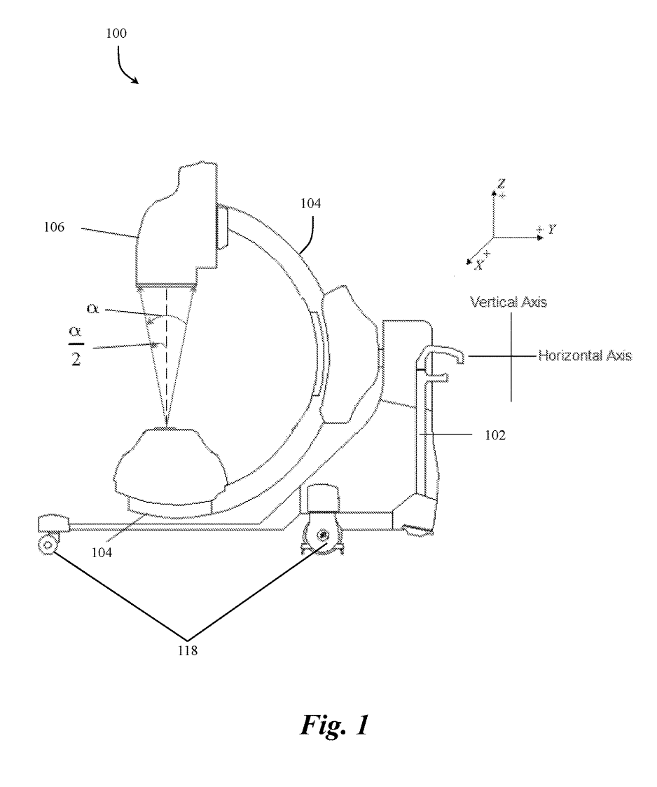

Conventional cone beam computed tomography imaging procedures (e.g., interventional radiology) are implemented by mounting a CBCT scanner on a C-arm apparatus. An example of a conventional C-arm apparatus configured for a CBCT implementation is depicted in FIG. 1. In particular, FIG. 1 depicts the main components of a convention C-arm apparatus 100 configured with a CBCT scanner to be utilized during a interventional radiology or other three dimensional imaging procedure. The main components of the C-arm apparatus 100 include a movable stand 102, an imaging energy emitter 104 (e.g., radiation source, X-ray tube, etc.), and imaging receptor 106 (e.g., radiation detector, image intensifier, flat panel detector, etc.), and a patient table (not depicted) configured to hold a patient between the imaging energy emitter 104 and the imaging receptor 106. The imaging energy emitter 104 is configured to produce a radiation beam (e.g., X-ray beam) with an angle alpha (α) which has a half of the alpha angle (α/2), as depicted in FIG. 1. As would be appreciated by one skilled in the art, the imaging energy emitter 104 can include any kind of radiation sources utilized for imaging a patient. For example, the imaging energy emitter 104 can be electromagnetic radiation or x-radiation sources configured to produce X-rays. In operation, to acquire three-dimensional images, the C-arm apparatus 100 is rotated 180 degrees plus a distance of the alpha angle (α) in the Y-Z plane (half scan). During the rotation, the C-arm apparatus 100, specifically, the imaging receptor 106, captures a plurality of images to be transformed into a three-dimensional image (e.g., via an image processor).

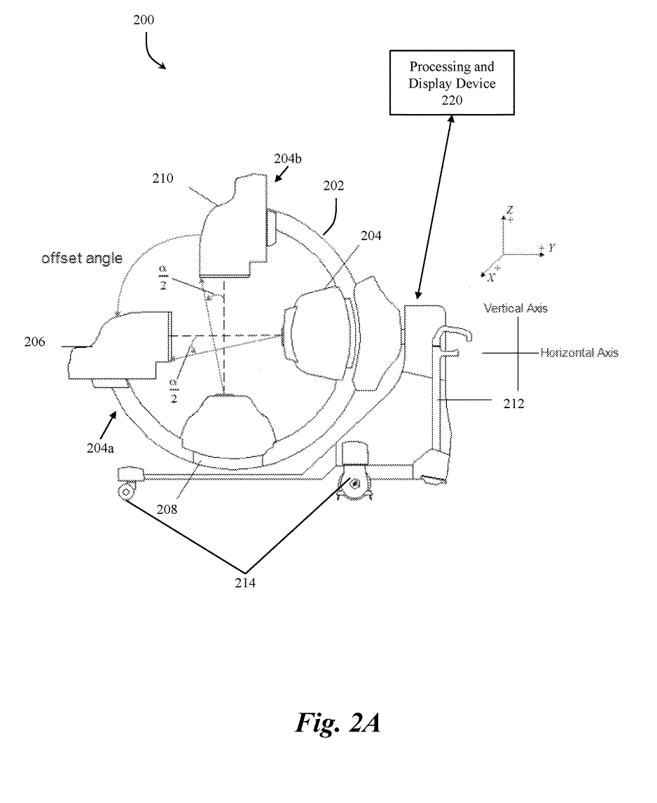

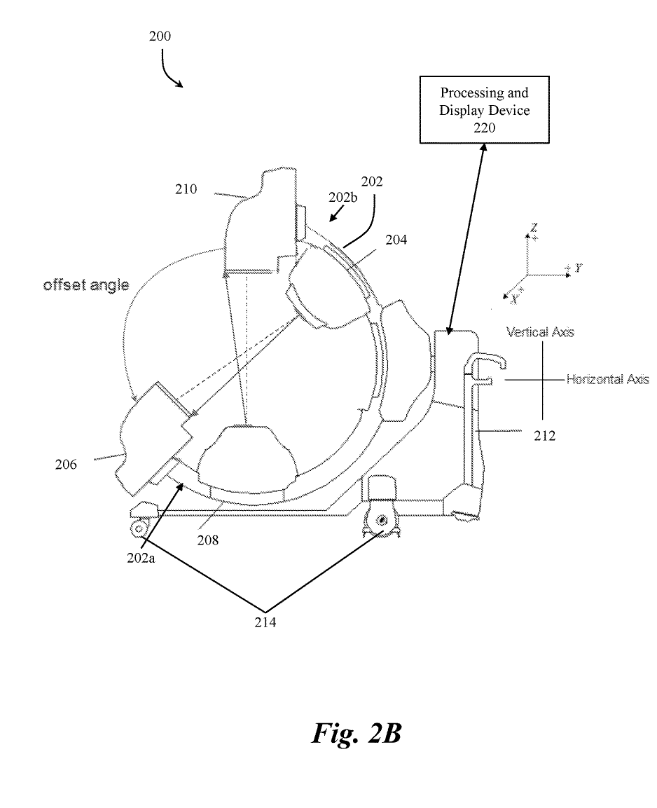

FIGS. 2A, 2B, 3, and 4 wherein like parts are designated by like reference numerals throughout, illustrate an example embodiment or embodiments of a quarter scan system and corresponding method for producing three-dimensional images using a mobile bi-planar imaging device, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

As utilized herein, the phrase “LT plane” refers to the mean or sagittal plane of a patient, and the phrase “AP plane” refers to the transverse or axial plane of a patient, which is perpendicular to the LT plane. Such terminology is utilized in compliance with conventional meanings in the field of medical imaging.

FIGS. 2A and 2B depict an example embodiment of a mobile bi-planar imaging device 200 for use in accordance with the present invention. In particular, FIGS. 2A and 2B depicts a mobile bi-planar imaging device 200 configured to perform three-dimensional imaging using a CBCT quarter scan technique. As would be appreciated by one skilled in the art, the bi-planar imaging device 200 can include any combination of imaging devices capable of being configured with the aspect of the present invention and is not limited to a mobile device. For example, the mobile bi-planar imaging device 200 is one of a ceiling or flooring mounted dual plane fluoroscopic system In accordance with an example embodiment of the present invention, the mobile bi-planar imaging device 200 is configured to perform a paradoxical pulse bi-planar synchronous real-time imaging (e.g., for fluoroscopic procedures) using a CBCT quarter scan technique. The mobile bi-planar imaging device 200 includes a support gantry 202 having a generally arc shape about an interior center focus point with a first terminal end 202a and a second terminal end 202b. The support gantry 202 is configured for mounting the various components for performing three-dimensional imaging during execution of the CBCT quarter scan technique.

The first imaging assembly includes a first imaging energy emitter 204 positioned opposite a first imaging receptor 206. In accordance with an example embodiment of the present invention, one of the first imaging energy emitter 204 and the first imaging receptor 206 is positioned and oriented at a first terminal end 202a of the support gantry 202, as depicted in FIGS. 2A and 2B. The positioning and orientation of the first imaging assembly, as depicted in FIGS. 2A and 2B, is configured to emit imaging energy (e.g., from the first imaging energy emitter 204) in an LT plane of a centrally located subject. FIGS. 2A and 2B depicts the first imaging receptor 206 at the first terminal end 202a of the support gantry 202, however as would be appreciated by one skilled in the art, the first imaging energy emitter 204 could be positioned at the first terminal end 202a with the first imaging receptor 206 positioned on the opposite side of the support gantry 202 without influencing the imaging process. In other words, the first imaging receptor 206 (shown in FIGS. 2A and 2B) can be positionally switched with the first imaging energy emitter 204 (shown in FIGS. 2A and 2B).

The second imaging assembly includes a second imaging energy emitter 208 positioned opposite a second imaging receptor 210. In accordance with an example embodiment of the present invention, one of the second imaging energy emitter 208 or the second imaging receptor 210 is positioned at the second terminal end 202b of the support gantry 202. The second imaging assembly is positioned and oriented, as depicted in FIGS. 2A and 2B, to emit imaging energy in an AP plane, perpendicular to the LT plane created by the first imaging assembly. In particular, FIGS. 2A and 2B depicts the second imaging receptor 210 at the second terminal end 202b of the support gantry 202, however as would be appreciated by one skilled in the art, the second imaging energy emitter 208 could be positioned at the second terminal end 202b with the second imaging receptor 210 positioned on the opposite side of the support gantry 202 without influencing the imaging process. In other words, the second imaging receptor 210 (shown in FIGS. 2A and 2B) can also be switched with the second imaging energy emitter 208 (shown in FIGS. 2A and 2B) in an optional arrangement.

Continuing with FIGS. 2A and 2B, the mobile bi-planar imaging device 200 also includes a control unit 212 configured to move and position the support gantry 202 to a desired location. In accordance with an example embodiment of the present invention, the support gantry 202 includes a plurality of wheels 214 to enable a user to push, pull, and pivot the mobile bi-planar imaging device 200 into a desired position via the control unit 212. The mobile bi-planar imaging device 200 includes or is otherwise in communication with a processing and display device 220 (such as the imaging control device discussed in U.S. Patent Application Publication No. 2016/0262712 incorporated herein by reference). The processing and display device 220 is configured to receive readouts from the imaging receptors 206, 210 and convert the readouts signal into a displayable format. In particular the processing and display device 220 displays the readouts as a three-dimensional image. For example, the imaging receptors 206, 210 can be thin film transistor (TFT) panels with a scintillation material layer configured to receive energy from visible photons to charge capacitors of pixel cells within the TFT panel. The charges for each of the pixel cells are readout as a voltage data value to the processing and display device 220. The signals received by the processing and display device 220 can be configured into three dimensional images utilizing any combination of methodologies known in the art. As would be appreciated by one skilled in the art, any type of imaging receptors 206, 210 could be used without departing from the scope of the present invention.

In accordance with an example embodiment of the present invention, the first imaging assembly and the second imaging assembly are each configured to rotate along the generally arc shape of the support gantry 202. As would be appreciated by one skilled in the art, the imaging assemblies can be rotated around the support gantry 202 through any combination of mechanical and manual processes. The rotation of the first imaging assembly causes one of the first imaging energy emitter 204 or the first imaging receptor 206 positioned at the first terminal end 202a of the support gantry 202 to rotate between the second imaging energy emitter 208 and the second imaging receptor 210 of the second imaging assembly at an offset angle between the first imaging receptor and the second imaging receptor of 0° to 180°. FIGS. 2A and 2B, depict examples of mechanical offset angle, which is between of the two imaging receptors 206, 210 on the support gantry 202. In convention G-Arm configurations, mechanical offset angle can only be set at 90°, as depicted in FIG. 2A. However, in the G-Arm configuration enabled by the present invention, mechanical offset angle can be set at any angle between 0° and 180°, as depicted in a varied angle provided in FIG. 2B.

In accordance with an example embodiment of the present invention, the first imaging energy emitter 204 and the second imaging energy emitter 208 are configured for producing divergent X-ray forming a cone for three dimensional CBCT imaging. In particular, the positioning of the imaging energy emitters 204, 208 enable the mobile bi-planar imaging device 200 to be utilized for real-time three dimensional imaging of a stationary subject (e.g., a patient). During the procedure, the first imaging assembly and the second imaging assembly (including the imaging energy emitters 204, 208 or CBCT scanners) rotate simultaneously around a subject (e.g., patient), positioned centrally within the support gantry 202. For example, the first imaging assembly and the second imaging assembly are rotated by implementing a motorized rack and pinion mechanism on the support gantry 202. While the first imaging assembly and the second imaging assembly are rotating, energy emitted by the imaging energy emitters 204, 208 is captured by the first imaging receptor 206 and second imaging receptor 210, respectively. The captured energy is converted into a plurality of raw image data (each individual set of raw data is captured at periodic rotation points) to be transmitted and transformed into three-dimensional images (e.g., digital volume) by the processing and display device 220.

In accordance with an example embodiment of the present invention, the imaging energy emitters 204, 208 produce radiation beams (e.g., X-ray beam) with an angle alpha (α) which have a half of the alpha angle (α/2). As would be appreciated by one skilled in the art, the imaging energy emitters 204, 208 can include any kind of radiation sources utilized for CBCT imaging a patient. For example, the imaging energy emitters 204, 208 can be electromagnetic radiation or x-radiation sources configured to produce X-rays.

In an exemplary example operation, the apparatus performs a three-dimensional image scan by positioning the first imaging assembly and the second imaging assembly at locations around the subject. The location of the first imaging assembly and the second imaging assembly are mechanically offset from one another to create a mechanical offset angle between the first imaging receptor and the second energy emitter of 90 degree plus a distance of half a fan beam angle produced by energy emissions of the first imaging energy emitter 204 and the second imaging energy emitter 208. In particular, in accordance with an example embodiment of the present invention, to acquire three-dimensional images, both of the imaging energy emitters 204, 208 (and corresponding imaging receptors 206, 210) are rotated 90 degrees plus one half alpha angle in the Y-Z plane direction about the G-arm support gantry 202. Data is captured by the rotating imaging receptors 206, 210 and the three-dimensional image is formed by reconstructing the data (e.g., by the processing and display device 220) using any methodology known in the art. By limiting the rotation of the first imaging assembly and the second imaging assembly to 90 degrees plus one half the alpha angle (e.g., half the angle of the X-ray beam produced by the imaging energy emitters 204, 208), the present invention provides a convenient and time-saving three-dimensional imaging methodology to be utilized by doctors during surgeries. Additionally, the half fan beam angle provides a benefit of consistent back-projection weighting during the reconstruction process (e.g., at the processing and display device 220).

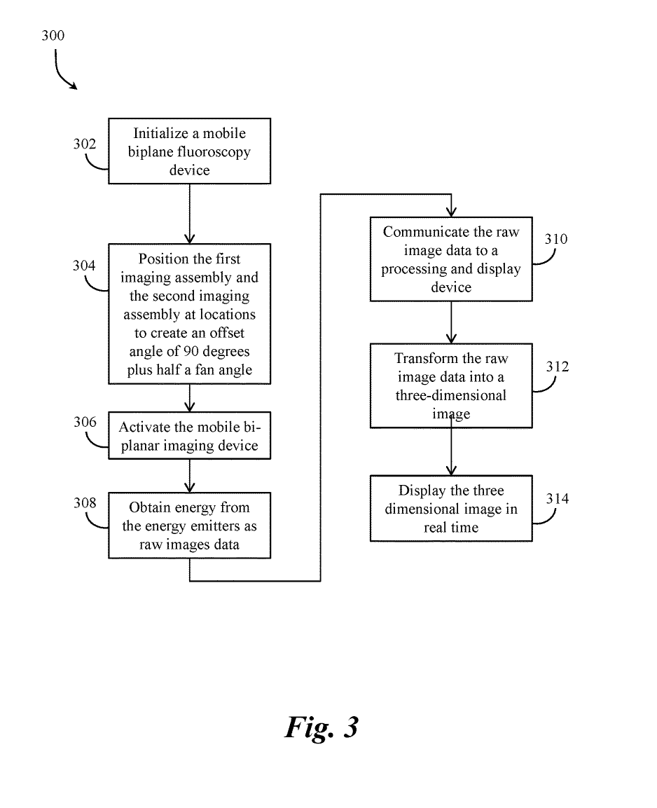

FIG. 3 shows an exemplary flow chart depicting an example method of operation of the present invention. Specifically, FIG. 3 depicts an exemplary flow chart showing the method operation of the mobile bi-planar imaging device 200 discussed with respect to FIGS. 2A and 2B. In particular, FIG. 3 depicts the method 300 for conducing three-dimensional cone beam computed tomography imaging with a mobile bi-planar imaging device 200. As discussed with respect to FIGS. 2A and 2B, the mobile bi-planar imaging device 200 includes a support gantry 202 having a generally arc shape about an interior center focus point with a first terminal end 202a and a second terminal end 202b. The mobile bi-planar imaging device 200 also includes a first imaging assembly positioned on the support gantry 202 and configured to rotate along the arc shape of the support gantry 202, the first imaging assembly including a first imaging energy emitter 204 positioned opposite a first imaging receptor 206, such that one of the first imaging energy emitter 204 or the first imaging receptor 206 is positioned at the first terminal end 202a of the support gantry 202. The mobile bi-planar imaging device 200 further includes a second imaging assembly positioned on the support gantry 202, the second imaging assembly includes a second imaging energy emitter 208 positioned opposite a second imaging receptor 210, such that one of the second imaging energy emitter 208 or the second imaging receptor 210 is positioned at the second terminal end 202b of the support gantry 202. Lastly, the mobile bi-planar imaging includes a control unit 212 that directs movement and positioning of the support gantry 202.

The method 300 starts at step 302 which includes initializing a mobile bi-planar imaging device 200. The initialization of the mobile bi-planar imaging device 200 can include positioning the device 200, positioning a subject on a table centered within the support gantry 202 of the device 200, with the imaging energy emitter(s) (e.g., imaging energy emitter 204, 208) of the device 200 directed at the subject.

At step 304 the first imaging assembly and the second imaging assembly are positioned at locations to create an offset angle between the first imaging receptor of the first imaging assembly and the second energy emitter of the second imaging assembly with a mechanical offset of 90 degrees plus half a fan beam angle produced by energy emissions of the first energy emitter and the second energy emitter. In accordance with an example embodiment of the present invention, the offset angle is 90 degree plus half of a fan beam angle. The fan beam angle is calculated by the detector width and the distance of source to detector. Positioning the first imaging assembly causes one of the first imaging energy emitter or the first imaging receptor positioned at the first terminal end of the support gantry to rotate between the second imaging energy emitter and the second imaging receptor of the second imaging assembly at an offset angle between the first imaging receptor and the second imaging receptor of 0 degrees to 180 degrees.

At step 306 the mobile bi-planar imaging device 200 is activated with a subject patient positioned between the first imaging assembly and the second imaging assembly. The activation of the mobile bi-planar imaging device 200 causes the CBCT imaging to being and the two imaging assembles rotate and the imaging energy emitters begin emitting energy (e.g., radiation). At step 308 the first imaging receptor and the second imaging receptor obtain energy from the energy emitters as raw images data of the subject patient. At step 310 communicate the raw image data to a processing and display device. At step 312 the raw image data is transformed, by the processing and display device, into a three-dimensional image of the subject patient. At step 314 the three-dimensional image is displayed on a display in real time. As would be appreciated by one skilled in the art, processes in steps 306-314 can be carried out through any combination of methods and systems known in the art.

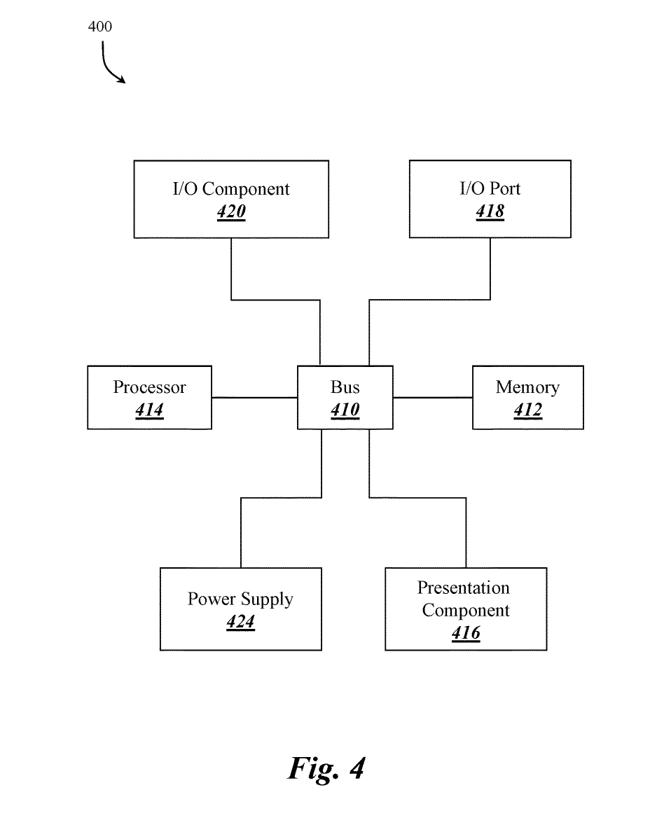

Any suitable computing device can be used to implement the computing devices 120 and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 400 is depicted in FIG. 4. The computing device 400 is merely an illustrative example of a suitable computing environment and in no way limits the scope of the present invention. A “computing device,” as represented by FIG. 4, can include a “workstation,” a “server,” a “laptop,” a “desktop,” a “hand-held device,” a “mobile device,” a “tablet computer,” or other computing devices, as would be understood by those of skill in the art. Given that the computing device 400 is depicted for illustrative purposes, embodiments of the present invention may utilize any number of computing devices 400 in any number of different ways to implement a single embodiment of the present invention. Accordingly, embodiments of the present invention are not limited to a single computing device 400, as would be appreciated by one with skill in the art, nor are they limited to a single type of implementation or configuration of the example computing device 400.

The computing device 400 can include a bus 410 that can be coupled to one or more of the following illustrative components, directly or indirectly: a memory 412, one or more processors 414, one or more presentation components 416, input/output ports 418, input/output components 420, and a power supply 424. One of skill in the art will appreciate that the bus 410 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 4 is merely illustrative of an exemplary computing device that can be used to implement one or more embodiments of the present invention, and in no way limits the invention.

The computing device 400 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 400.

The memory 412 can include computer-storage media in the form of volatile and/or nonvolatile memory. The memory 412 may be removable, non-removable, or any combination thereof. Exemplary hardware devices are devices such as hard drives, solid-state memory, optical-disc drives, and the like. The computing device 400 can include one or more processors that read data from components such as the memory 412, the various I/O components 416, etc. Presentation component(s) 416 present data indications to a user or other device. Exemplary presentation components include a display device, speaker, printing component, vibrating component, etc.

The I/O ports 418 can enable the computing device 400 to be logically coupled to other devices, such as I/O components 420. Some of the I/O components 420 can be built into the computing device 400. Examples of such I/O components 420 include a microphone, joystick, recording device, game pad, satellite dish, scanner, printer, wireless device, networking device, and the like.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method for conducing three-dimensional cone beam computed tomography imaging with a bi-planar imaging device, the method comprising:
initializing the bi-planar imaging device, the device comprising:
a support gantry having a generally arc shape about an interior center focus point with a first terminal end and a second terminal end;
a first imaging assembly positioned on the support gantry and configured to rotate along the generally arc shape of the support gantry, the first imaging assembly comprising a first imaging energy emitter positioned opposite a first imaging receptor, wherein one of the first imaging energy emitter or the first imaging receptor is positioned at the first terminal end of the support gantry;
a second imaging assembly positioned on the support gantry, the second imaging assembly comprising a second imaging energy emitter positioned opposite a second imaging receptor, wherein one of the second imaging energy emitter or the second imaging receptor is positioned at the second terminal end of the support gantry; and
a control unit that directs movement and positioning of the support gantry;
positioning the first imaging assembly and the second imaging assembly at locations to create an offset angle between the first imaging receptor of the first imaging assembly and the second imaging energy emitter of the second imaging assembly with a mechanical offset of 90 degrees plus half a fan beam angle produced by energy emissions of the first imaging energy emitter and the second imaging energy emitter; and
activating the bi-planar imaging device with a subject patient positioned between the first imaging assembly and the second imaging assembly;
obtaining, by the first imaging receptor and the second imaging receptor, raw image data of the subject patient;
communicating the raw image data to a processing and display device;
transforming the raw image data of the subject patient, by the processing and display device, into a three-dimensional image of the subject patient; and
displaying the three-dimensional image on a display.

2. The method of claim 1, wherein positioning the first imaging assembly causes one of the first imaging energy emitter or the first imaging receptor positioned at the first terminal end of the support gantry to rotate between the second imaging energy emitter and the second imaging receptor of the second imaging assembly at an offset angle between the first imaging receptor and the second imaging receptor of 0 degrees to 180 degrees.

3. The method of claim 1, wherein the first imaging assembly is positioned and oriented to emit imaging energy in an LT plane and the second imaging assembly is positioned and oriented to emit imaging energy in an AP plane, perpendicular to the LT plane.

4. The method of claim 1, wherein the first imaging assembly is positioned and oriented to emit imaging energy in an AP plane and the second imaging assembly is positioned and oriented to emit imaging energy in an LT plane, perpendicular to the AP plane.

5. The method of claim 1, wherein the first imaging receptor and the second imaging receptor are one of an image intensifier or a flat panel detector.

6. The method of claim 1, wherein the first imaging energy emitter and the second imaging energy emitter are X-ray sources configured to produce X-ray beams.

7. The method of claim 1, wherein the bi-planar imaging device is one of a ceiling or flooring mounted dual plane fluoroscopic system.

8. A bi-planar imaging apparatus, comprising:
a support gantry having a generally arc shape about an interior center focus point with a first terminal end and a second terminal end;
a first imaging assembly positioned on the support gantry and configured to rotate along the generally arc shape of the support gantry, the first imaging assembly comprising a first imaging energy emitter positioned opposite a first imaging receptor, wherein one of the first imaging energy emitter or the first imaging receptor is positioned at the first terminal end of the support gantry;
a second imaging assembly positioned on the support gantry, the second imaging assembly comprising a second imaging energy emitter positioned opposite a second imaging receptor, wherein one of the second imaging energy emitter or the second imaging receptor is positioned at the second terminal end of the support gantry; and
a control unit that directs movement and positioning of the support gantry;
wherein rotation of the first imaging assembly causes the one of the first imaging energy emitter or the first imaging receptor positioned at the first terminal end of the support gantry to rotate between the second imaging energy emitter and the second imaging receptor of the second imaging assembly at an offset angle between the first imaging receptor and the second imaging receptor of no 180°;
wherein the apparatus performs a three-dimensional image scan by positioning of the first imaging assembly and the second imaging assembly at locations to create the offset angle between the first imaging receptor of the first imaging assembly and the second imaging energy emitter of the second imaging assembly with a mechanical offset of 90 degree plus half a fan beam angle produced by energy emissions of the first imaging energy emitter and the second imaging energy emitter.

9. The apparatus of claim 8, wherein the first imaging assembly is positioned and oriented to emit imaging energy in an LT plane and the second imaging assembly is positioned and oriented to emit imaging energy in an AP plane, perpendicular to the LT plane.

10. The apparatus of claim 8, wherein the first imaging assembly is positioned and oriented to emit imaging energy in an AP plane and the second imaging assembly is positioned and oriented to emit imaging energy in an LT plane, perpendicular to the AP plane.

11. The apparatus of claim 8, wherein the first imaging receptor and the second imaging receptor are one of an image intensifier or a flat panel detector.

12. The apparatus of claim 8, wherein the first imaging energy emitter and the second imaging energy emitter are X-ray sources configured to produce X-ray beams.

13. The apparatus of claim 8, wherein the bi-planar imaging apparatus is mounted on a G-arm system.
